(19) United States
(12) Patent Application Publication
(10) Pub. No.: US 2015/0014874 A1
(43) Pub. Date: Jan. 15, 2015

(54) METHOD AND APPARATUS FOR DETERMINING A LIQUID LEVEL IN A HUMIDIFIED PRESSURE SUPPORT DEVICE

(71) Applicant: KONINKLIJKE PHILIPS N.V., EINDHOVEN (NL)

(72) Inventor: Jeffrey Ronald Winski, Irwin, PA (US)

(21) Appl. No.: 14/379,604

(22) PCT Filed: Feb. 21, 2013
(86) PCT No.: PCT/IB2013/051416

§ 371 Date: Aug. 19, 2014

Related U.S. Application Data
(60) Provisional application No. 61/605,240, filed on Mar. 1, 2012.

(51) Int. Cl. A61M 16/16

(52) U.S. Cl. CPC A61M 16/16
USPC 261/128; 261/130

(57) ABSTRACT

The present disclosure pertains to a pressure support system configured to provide pressure support therapy to a subject, wherein the pressure support system is configured to determine a volume of liquid held by a humidifier. The liquid volume determination is based on the temperature of the liquid in the humidifier, the temperature of the heater in the humidifier, and/or the temperature of a component of the heater (e.g., a heating element), subsequent to modulation of the rate at which energy is dissipated into the liquid by a heater. Applying a known amount of energy for a known amount of time (power) increases the temperature of the liquid in the humidifier. The amount of liquid in the humidifier can be determined from the applied power and the rise in liquid temperature, heater temperature, and/or heater component temperature over time because the rise in liquid temperature over time is proportional the volume of liquid in the humidifier.

### BACKGROUND

1. Field

The present disclosure pertains to a pressure support system configured to provide pressure support therapy to a subject, wherein the pressure support system is configured to determine a volume of liquid held by a humidifier.

2. Description of the Related Art

Pressure support systems that provide pressure support therapy to the airway of a subject are known. Some conventional pressure support systems include humidifiers configured to control the level of humidity of gas provided to the subject during pressure support therapy.

Humidifiers are commonly used with ventilators, pressure support systems, and other respiratory therapy devices to add humidity to the gas being supplied to a subject. The humidity added to the gas supplied to the subject by a conventional ventilator or pressure support system is typically monitored and/or controlled in a feedback loop to provide a consistent humidity level. Typically, these systems are configured to determine a target humidity output and set a humidifier heater temperature to achieve the target humidity. Such systems do not take into account the amount of liquid in the humidifier, sometimes resulting in the humidifier running out of liquid. A humidifier that runs out of liquid while the pressure support system is still operating may result in delivery of a hot, dry gas to the patient, leading to a dry and sore mucus membrane that can lead to other medical issues.

### SUMMARY

Accordingly, one or more aspects of the present disclosure relate to a pressure support system configured to provide pressure support to a subject. In some embodiments, the pressure support system comprises a pressure generator, a humidifier, one or more temperature sensors, a heater, and/or one or more processors. In some embodiments, the one or more processors comprise a temperature module, a heater control module, and/or a liquid volume module. The pressure generator is configured to generate a pressurized flow of breathable gas for delivery to the airway of a subject. The humidifier is configured to hold a volume of liquid and humidify the pressurized flow of breathable gas. The one or more temperature sensors are configured to generate one or more output signals conveying information related to the temperature of the liquid in the humidifier, the temperature of the heater, and/or the temperature of a component of the heater. The heater is configured to controllably dissipate energy into the liquid to elevate the temperature of the liquid within the humidifier such that vapor formed from the heated liquid humidifies the pressurized flow of breathable gas. The temperature module is configured to determine the temperature of the liquid held by the humidifier, the temperature of the heater, and/or the temperature of the component of the heater based on the output signals generated by the one or more temperature sensors. The heater control module is configured to modulate the rate at which energy is dissipated into the liquid by the heater. The liquid volume module is configured to determine the volume of the liquid held by the humidifier based on the temperature of the liquid in the humidifier, the temperature of the heater, and/or the temperature of a component of the heater subsequent to modulation of the rate at which energy is dissipated into the liquid by the heater.

Yet another aspect of the present disclosure relates to a method of generating a pressurized flow of breathable gas for delivery to the airway of a subject. The method comprises generating a pressurized flow of breathable gas for delivery to the airway of a subject; holding a volume of liquid in a humidifier; generating one or more output signals conveying information related to the temperature of the liquid in the humidifier, the temperature of the heater, and/or the temperature of a component of the heater; controllably dissipating energy into the liquid to elevate the temperature of the liquid within the humidifier such that vapor formed from the heated liquid humidifies the pressurized flow of breathable gas; determining the temperature of the liquid held by the humidifier, the temperature of the heater, and/or the temperature of the component of the heater based on the output signals conveying information related to the temperature of the liquid in the humidifier, the temperature of the heater, and/or the temperature of the component of the heater; modulating the rate at which energy is dissipated into the liquid; and determining the volume of the liquid held by the humidifier based on the temperature of the liquid in the humidifier, the temperature of the heater, and/or the temperature of the component of the heater subsequent to modulation of the rate at which energy is dissipated into the liquid.

Still another aspect of the present disclosure relates to a pressure support system configured to provide pressure support to a subject. The pressure support system comprises means to generate a pressurized flow of breathable gas for delivery to the airway of a subject; means to hold a volume of liquid and humidify the pressurized flow of breathable gas; and means to execute computer program modules. The means to humidify comprises means to generate one or more output signals conveying information related to the temperature of the liquid in the means to humidify, the temperature of a means to controllably dissipate energy into the liquid in the means to humidify, and/or the temperature of a component of the means to dissipate energy, and means to controllably dissipate energy into the liquid to elevate the temperature of the liquid within the means to humidify such that vapor formed from the heated liquid humidifies the pressurized flow of breathable gas. The computer program modules comprise means to determine the temperature of the liquid held by the means to humidify, the temperature of the means to controllably dissipate energy, and/or the temperature of the component of the means to controllably dissipate energy based on the output signals generated by the means to convey information related to the temperature of the liquid in the means to humidify, the temperature of the means to controllably dissipate energy, and/or the component of the means to controllably dissipate energy; means to modulate the rate at which energy is dissipated into the liquid by the means to controllably dissipate energy into the liquid; and means to determine the volume of the liquid held by the means to humidify based on the temperature of the liquid in the means to humidify, the temperature of the means to controllably dissipate energy, and/or the temperature of the component of the means to controllably dissipate energy subsequent to modulation of the rate at which energy is dissipated into the liquid by the means to controllably dissipate energy into the liquid.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 is a pressure support system configured to provide pressure support to a subject; and

FIG. 2 is a method for providing pressure support to a subject.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

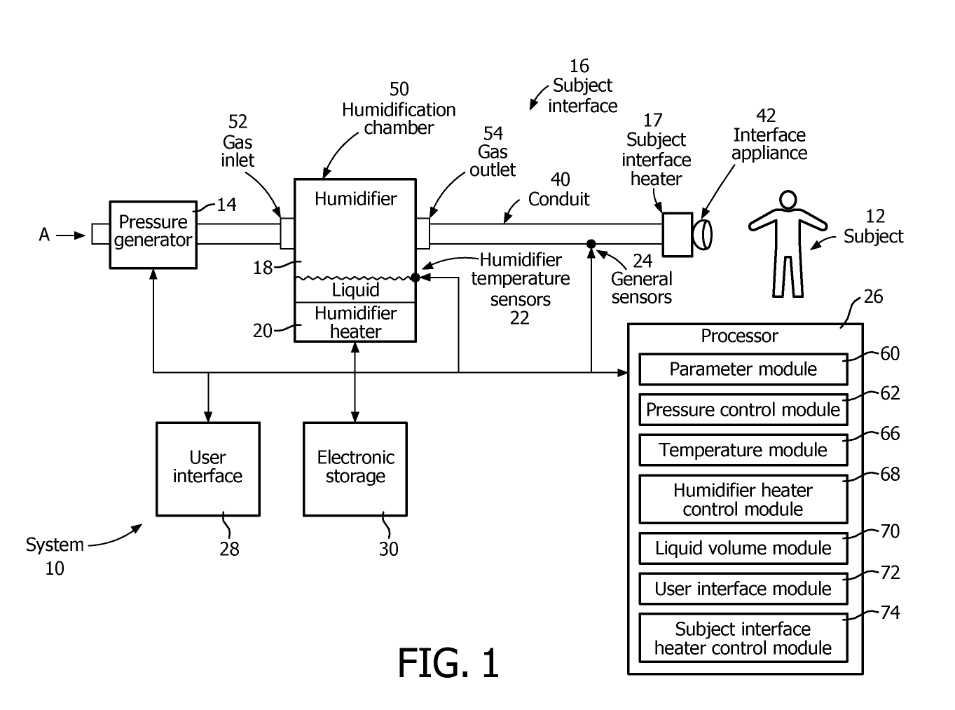

FIG. 1 schematically illustrates a pressure support system 10 configured to provide pressure support therapy to a subject 12. Pressure support system 10 is configured to provide the pressure support therapy in the form of a flow of gas that is delivered to the airway of subject 12. The pressure support therapy may be dynamic in that one or more parameters of the flow of gas generated by pressure support system 10 may be adjusted based on detection of one or more parameters. For example, pressure of the flow of gas may be increased based on changes to one or more parameters that indicate a respiratory event (e.g., an apnea, snoring, etc.).

Pressure support system 10 is configured to determine the volume of the liquid held by the humidifier. The liquid volume determination is based on the temperature of the liquid in the humidifier and/or the humidifier heater temperature subsequent to modulation of the rate at which energy is dissipated into the liquid by the heater. Modulation may be performed during operation of the pressure support system and/or at times when the system is not generating pressure support. Applying a known amount of energy for a known amount of time (power) increases the temperature of the liquid and/or a heating element in the heater, for example, in the humidifier. The amount of liquid in the humidifier can be determined from the applied power and the rise in liquid temperature and/or heating element temperature over time, because the rise in liquid and/or heating element temperature over time is proportional the volume of liquid in the humidifier.

In one embodiment, pressure support system 10 comprises one or more of a pressure generator 14, a subject interface 16, a subject interface heater 17, a humidifier 18, a humidifier heater 20, one or more humidifier temperature sensors 22, one or more general sensors 24, a processor 26, a user interface 28, electronic storage 30, and/or other components.

In some embodiments, pressure generator 14 is configured to generate a flow of gas for delivery to the airway of a subject 12. Pressure generator 14 may control one or more parameters of the flow of gas (e.g., flow rate, pressure, volume, temperature, gas composition, etc.) for therapeutic purposes, and/or for other purposes. Byway of a no n-limiting example, pressure generator 14 maybe configured to control the flow rate and/or pressure of the flow of gas to provide pressure support to the airway of the subject.

Pressure generator 14 receives a flow of gas from a gas source, such as the ambient atmosphere, as indicated by arrow A and elevates the pressure of that gas for delivery to the airway of a patient. Pressure generator 14 is any device, such as, for example, a pump, blower, piston, or bellows, that is capable of elevating the pressure of the received gas for delivery to a patient. The present disclosure also contemplates that gas other than ambient atmospheric air may be introduced into system 10 for delivery to the patient. In such embodiments, a pressurized canister or tank of gas containing air, oxygen, and/or another gas may supply the intake of pressure generator 14. In some embodiments, pressure generator 14 need not be provided, but instead the gas may be pressurized by the pressure of the canister and/or tank of pressurized gas itself.

In one embodiment, pressure generator 14 is a blower that is driven at a substantially constant speed during the course of the pressure support treatment to provide the gas in system 10 with a substantially constant elevated pressure and/or flow rate. Pressure generator 14 may comprise a valve for controlling the pressure/flow of gas. The present disclosure also contemplates controlling the operating speed of the blower, either alone or in combination with such a valve, to control the pressure/flow of gas provided to the patent. An example of a pressure support system suitable for use in the present disclosure is described in U.S. Pat. No. 6,105,575, hereby incorporated by reference in its entirety.

Subject interface 16 is configured to deliver the pressurized flow of breathable gas to the airway of subject 12. As such, subject interface 16 comprises conduit 40, interface appliance 42, and/or other components. Conduit 40 is configured to convey the pressurized flow of gas to interface appliance 42. Interface appliance 42 is configured to deliver the flow of gas to the airway of subject 12. In some embodiments, interface appliance 42 is non-invasive. As such, interface appliance 42 non-invasively engages subject 12. Non-invasive engagement comprises removably engaging an area (or areas) surrounding one or more external orifices of the airway of subject 12 (e.g., nostrils and/or mouth) to communicate gas between the airway of subject 12 and interface appliance 42. Some examples of non-invasive interface appliance 42 may comprise, for example, a nasal cannula, a nasal mask, a nasal/oral mask, a full face mask, a total face mask, or other interface appliances that communicate a flow of gas with an airway of a subject. The present disclosure is not limited to these examples, and contemplates delivery of the flow of gas to the subject using any interface appliance.

Although subject interface 16 is illustrated in FIG. 1 as a single-limbed interface for the delivery of the flow of gas to the airway of the subject, this is not intended to be limiting. The scope of this disclosure comprises double-limbed circuits having a first limb configured to both provide the flow of gas to the airway of the subject, and a second limb configured to selectively exhaust gas (e.g., to exhaust exhaled gases).

Subject interface heater 17 is configured to controllably heat the pressurized flow of breathable gas in subject interface 16. Subject interface heater 17 is illustrated in FIG. 1 at a single location within (or in communication with) conduit 40, near interface appliance 42, and/or within interface appliance 42. The illustrated position of subject interface heater 17 is not intended to be limiting. Subject interface heater 17 may be located in any position that allows it to controllably heat the pressurized flow of breathable gas in subject interface 16. Subject interface heater 17 may be configured to heat the pressurized flow of breathable gas continuously along the entire length of conduit 40. Subject interface heater 17 may be configured to heat the pressurized flow of breathable gas by dissipating electrical current (e.g., resistive heating). Subject interface heater 17 may comprise one or more of a heating coil, a heating jacket, heating tape, and/or other heating devices. Subject interface heater 17 may be configured to heat the gas in subject interface 16 directly and/or indirectly. In some embodiments, a heating coil may be positioned within conduit 40 in fluid communication with the pressurized flow of breathable gas to directly heat the gas flow. In some embodiments, a heating jacket may be placed around conduit 40 to heat the flow of gas indirectly by transferring heat through the wall of conduit 40.

Humidifier 18 is configured to humidify the flow of gas in system 10. Humidifier 18 may comprise a humidification chamber 50, a gas inlet 52, a gas outlet 54, a humidifier heater 20, and/or other components. In one embodiment, humidifier 18 is a warm mist humidifier (e.g., a vaporizer) configured to generate water vapor by heating liquid held within humidifier 18 via humidifier heater 20. Humidifier 18 may comprise an inductive heater configured to heat the liquid held within humidifier 18 via inductive heating. Humidifier 18 is configured such that the flow of gas is received from pressure generator 14 by humidifier 18 through gas inlet 52 and is humidified within humidification chamber 50 by the water vapor before being released from humidifier 18 through gas outlet 54. In one embodiment, gas outlet 54 is connected with subject interface 16 such that the humidified flow of gas is delivered to the airway of subject 12 through subject interface 16.

Heater 20 is configured to controllably dissipate energy through a heating element, for example, into the liquid held by humidifier 18 to elevate the temperature of the liquid within humidifier 18 such that vapor formed from the heated liquid humidifies the pressurized flow of breathable gas. Humidifier heater 20 may be configured to heat the liquid in humidifier 20 by dissipating electrical current (e.g., resistive heating). In some embodiments, humidifier heater 20 is positioned at the bottom of humidifier 18 in proximity to the liquid in humidifier 18. The energy dissipated by humidifier heater 20 is dispensed directly into the liquid in humidifier 18. This dissipation of energy by humidifier heater 20 into the liquid vaporizes the liquid. The illustrated position of humidifier heater 20 is not intended to be limiting. Humidifier heater 20 may be located in any position that allows it to controllably dissipate energy into some and/or all of the liquid held by humidifier 18. The illustration of humidifier heater 20 as an individual element should not be viewed as limiting. In some embodiments, humidifier heater 20 includes a plurality of elements arranged near each other and/or at disparate locations.

One or more humidifier temperature sensors 22 are configured to generate output signals conveying information related to the temperature of the liquid in humidifier 18, the temperature of humidifier heater 20, and/or the temperature of a component of humidifier heater 20 (e.g., a heating element). Humidifier temperature sensors 22 may comprise one or more sensors that measure such parameters directly (e.g., through communication with the liquid in humidifier 18 and/or a heating element in humidifier heater 20). Humidifier temperature sensors 22 may comprise one or more sensors that generate output signals related to a current temperature in humidifier 18 indirectly. For example, humidifier temperature sensors 22 may comprise one or more sensors configured to generate an output based on an operating parameter of humidifier 18 and/or humidifier heater 20 (e.g., a current drawn, voltage, and/or other operating parameters), and/or other sensors. Although humidifier temperature sensors 22 are illustrated in FIG. 1 at a single location in system 10, this is not intended to be limiting. Humidifier temperature sensors 22 may comprise sensors disposed in a plurality of locations, such as for example, at various locations within (or in communication with) humidifier 18, and/or other locations.

One or more general sensors 24 are configured to generate output signals conveying information related to one or more parameters of the gas within system 10. The one or more parameters of the gas within system 10 may comprise gas parameters related to the pressurized flow of breathable gas, breathing parameters related to respiration of subject 12, and/or other parameters. General sensors 24 may comprise one or more sensors that measure such parameters directly (e.g., through fluid communication with the flow of gas in interface appliance 42). General sensors 24 may comprise one or more sensors that generate output signals related to the one or more parameters indirectly. For example, general sensors 24 may comprise one or more sensors configured to generate an output based on an operating parameter of pressure generator 14 (e.g., a valve driver or motor current, voltage, rotational velocity, and/or other operating parameters), and/or other sensors.

The one or more gas parameters of the pressurized flow of breathable gas may comprise, for example, one or more of a flow rate, a volume, a pressure, humidity, temperature, acceleration, velocity, and/or other gas parameters.

Breathing parameters related to the respiration of subject 12 may comprise a tidal volume, a timing (e.g., beginning and/or end of inhalation, beginning and/or end of exhalation, etc.), a respiration rate, a duration (e.g., of inhalation, of exhalation, of a single breathing cycle, etc.), respiration frequency, and/or other breathing parameters. The one or more breathing parameters of subject 12 may comprise other parameters that provide information about the breathing of the subject. For example, general sensors 24 may comprise a transducer configured to detect acoustic waves transmitted to pressure support system 10 through subject interface 16. These acoustic waves may convey information related to respiratory effort of the subject, and/or the noise generated by the subject during respiration (e.g., during snoring).

Although general sensors 24 are illustrated in FIG. 1 at a single location in system 10, this is not intended to be limiting. General sensors 24 may comprise sensors disposed in a plurality of locations, such as for example, at various locations within (or in communication with) conduit 40, within pressure generator 14, within humidifier 18, within (or in communication with) interface appliance 42, and/or other locations.

Processor 26 is configured to provide information processing capabilities in system 10. As such, processor 26 may comprise one or more of a digital processor, an analog processor, a digital circuit designed to process information, an analog circuit designed to process information, a state machine, and/or other mechanisms for electronically processing information. Although processor 26 is shown in FIG. 1 as a single entity, this is for illustrative purposes only. In some implementations, processor 26 may comprise a plurality of processing units. These processing units may be physically located within the same device (e.g., humidifier 18), or processor 26 may represent processing functionality of a plurality of devices operating in coordination.

As shown in FIG. 1, processor 26 is configured to execute one or more computer program modules. The one or more computer program modules may comprise one or more of a parameter module 60, a pressure control module 62, a temperature module 66, a humidifier heater control module 68, a liquid volume module 70, a user interface module 72, subject interface heater control module 74, and/or other modules. Processor 26 may be configured to execute modules 60, 62, 66, 68, 70, 72 and/or 74 by software; hardware; firmware; some combination of software, hardware, and/or firmware; and/or other mechanisms for configuring processing capabilities on processor 26.

It should be appreciated that although modules 60, 62, 66, 68, 70, 72 and/or 74 are illustrated in FIG. 1 as being co-located within a single processing unit, in implementations in which processor 26 comprises multiple processing units, one or more of modules 60, 62, 66, 68, 70, 72 and/or 74 may be located remotely from the other modules. The description of the functionality provided by the different modules 60, 62, 66, 68, 70, 72 and/or 74 described below is for illustrative purposes, and is not intended to be limiting, as any of modules 60, 62, 66, 68, 70, 72 and/or 74 may provide more or less functionality than is described. For example, one or more of modules 60, 62, 66, 68, 70, 72 and/or 74 may be eliminated, and some or all of its functionality may be provided by other modules 60, 62, 66, 68, 70, 72 and/or 74. As another example, processor 26 maybe configured to execute one or more additional modules that may perform some or all of the functionality attributed below to one of modules 60, 62, 66, 68, 70, 72 and/or 74.

Parameter module 60 is configured to determine one or more parameters within system 10. The one or more parameters within system 10 may comprise gas parameters related to the pressurized flow of breathable gas, breathing parameters related to respiration of subject 12, and/or other parameters. Parameter module 60 is configured to determine the one or more parameters based on the output signals of general sensors 24. The information determined by parameter module 60 may be used for controlling pressure generator 14, controlling humidifier 18, and/or for other uses.

Pressure control module 62 is configured to control pressure generator 14 to generate the flow of gas in accordance with the therapy regime. By way of non-limiting example, processor 26 may control pressure generator 14 such that the pressure support provided to the subject via the flow of gas comprises non-invasive ventilation, positive airway pressure support, continuous positive airway pressure support, bi-level support, BiPAP®, and/or other types of pressure support therapy.

Temperature module 66 is configured to determine the temperature of the liquid held by humidifier 18, the temperature of humidifier heater 20, and/or the temperature of a component of humidifier heater 20 (e.g., a heating element). Temperature module 66 is configured to determine the temperature based on the output signals generated by humidifier temperature sensors 22.

Humidifier heater control module 68 is configured to modulate the rate at which energy is dissipated into the liquid held in humidifier 18 by humidifier heater 20. In some embodiments, humidifier heater control module 68 is configured such that modulation of the rate at which energy is dissipated to the liquid comprises an increase in the rate, a decrease in the rate, and/or other modulations (e.g., temporary modulations). In some embodiments, humidifier heater control module 68 may be configured to modulate the power provided to heater 20 independent of the operation of pressure generator 14. In some embodiments, humidifier heater control module 68 may be configured to modulate the power provided to heater 20 at temperatures lower than the temperature necessary to induce the liquid to gas phase change in humidifier 18. For example, humidifier heater control module 68 may be configured to determine an initial liquid level in humidifier 18 by modulating the power provided to heater 20 before humidification begins, with pressure generator 14 turned off.

Humidifier heater control module 68 may control humidifier heater 20 in humidifier 18 to heat and/or vaporize fluid within humidifier 18 to adjust the amount of moisture added to the flow of gas within humidifier 18. The level of humidity to which the flow of gas is adjusted may be dictated by a therapy regime and/or selected by a user (e.g., the subject, a caregiver, a therapy decision-maker, etc.).

Humidifier heater control module 68 may be configured to modulate the rate at which energy is dissipated into the liquid held in humidifier 18 by humidifier heater 20 to facilitate determination of a liquid level at one or more individual time points, at programmed time intervals, continuously during a defined time period, upon start-up, and/or at other times during a usage session. In some embodiments, the timing of modulations may be determined at manufacture, determined by humidifier heater control module 68 based on information entered by a user via user interface 28, based on a determination of one or more use events, and/or by another method.

In some embodiments a use event may be determined by humidifier heater control module 68 and/or other modules. A use event may comprise one or more of expiration of a monitored period of time, an arousal event, a gas and/or breathing parameter change of some amount (e.g., indicating wakeful sleep by subject 12), and/or other use events. A use event may comprise an instantaneous occurrence (e.g. an arousal event), and/or conditions/circumstances that last for a period of time. In some embodiments, humidifier heater control module 68 may detect a use event when subject 12 wakes from sleep (instantaneous event). In some embodiments, humidifier heater control module 68 may detect a use event when subject 12 experiences wakeful sleep (e.g., movement, shallow breathing, etc.) over time (lasting condition).

In some embodiments, the timing (e.g., individual time points, programmed time intervals, etc.) of the modulations is determined based on one or more of the factors above. For example, a user may program humidifier heater control module 68 via user interface 28 to modulate the energy dissipation rate once every hour. Humidifier heater control module 68 may perform additional modulations based on information from parameter module 60 indicating that subject 12 woke up, for example.

Liquid volume module 70 is configured to determine the volume of the liquid held by humidifier 18. Liquid volume module 70 is configured to determine the volume based on the temperature of the liquid in humidifier 18, the temperature of humidifier heater 20, and/or the temperature of a component (e.g., a heating element) of humidifier heater 20 subsequent to modulation (by humidifier heater control module 68) of the rate at which energy is dissipated into the liquid by humidifier heater 20. In some embodiments, liquid volume module 70 may be configured to determine the volume of liquid held by humidifier 18 based on a change in one or more gas parameters (e.g., gas temperature, gas humidity) determined by parameter module 60 subsequent to modulation by humidifier heater control module 68 of the power provided to humidifier heater 20.

In some embodiments, liquid volume module 70 is configured to calculate the slope of the change in liquid temperature, heater temperature, heater component temperature, and/or gas parameter versus time, and to determine the volume of liquid proportional to the slope. Applying a known amount of energy for a known amount of time may increase the temperature of the liquid in humidifier 18. The amount of liquid in humidifier 18 may be determined from the known amount of applied energy over time and the rise in liquid temperature over time, because the rise in liquid temperature over time is proportional the volume of liquid in the humidifier. Similar relationships exist between the amount of applied energy over time and heater/heater component temperature, and between the amount of applied energy over time and a change in one or gas parameters (e.g., temperature, humidity). Subsequent to modulation by humidifier heater control module 68 of the power provided humidifier heater 20, the liquid and/or gas in humidifier 18 may act to absorb heat such that the amount of heat absorbed is proportional to the amount of liquid and/or gas in humidifier 18. The amount of heat absorbed may be proportional to the slope of the heater temp/gas parameter change over time.

In some embodiments, the liquid volume determination made by liquid volume module 70 may be utilized by humidifier heater control module 68 to determine when the amount of liquid in humidifier 18 is not sufficient to last through the entire current usage period. The algorithm used by heater control module 68 may include the liquid volume determination made by liquid volume module 70 as an input. Humidifier heater control module 68 may be configured to maintain a target liquid level in humidifier 18 by operating one or more valves to allow more liquid into humidifier 18.

User interface module 72 is configured to indicate information related to the determined volume of liquid in humidifier 18 to a user. The information may be indicated to a user via humidifier 18, user interface 28, and/or other devices. In some embodiments, user interface module 72 is configured to inform the user when the amount of liquid in humidifier 18 is not sufficient to last through the entire current usage period. User interface module 72 may be configured to communicate humidification status to the user. In some embodiments, the determined volume of liquid may be indicated to the user with an indicator light, a digital read out, and/or another method on humidifier 28, by way of numbers, letters, levels (e.g., low, medium, high), and/or other indications via user interface 28, and/or other methods. In short, any technique for indicating information related to the determined volume of liquid in humidifier 18 is contemplated by the present disclosure.

Subject interface heater control module 74 is configured to control subject interface heater 17 to maintain the temperature of the pressurized flow of breathable gas at a target temperature. In some embodiments, subject interface heater control module 74 may determine a target temperature based on information entered by a user through user interface 28, based on output signals from general sensors 24, based on information from humidifier heater control module 68, and/or other methods.

User interface 28 is configured to provide an interface between system 10 and subject 12 and/or other users through which subject 12 and/or other users may provide information to and receive information from system 10. Other users may comprise a caregiver, a doctor, and/or other users. This enables data, cues, results, and/or instructions and any other communicable items, collectively referred to as “information,” to be communicated between a user (e.g., subject 12) and one or more of pressure generator 14, processor 26, and/or other components of system 10. Examples of interface devices suitable for inclusion in user interface 28 comprise a keypad, buttons, switches, a keyboard, knobs, levers, a display screen, a touch screen, speakers, a microphone, an indicator light, an audible alarm, a printer, a tactile feedback device, and/or other interface devices. In one embodiment, user interface 28 comprises a plurality of separate interfaces. In one embodiment, user interface 28 comprises at least one interface that is provided integrally with pressure generator 14.

It is to be understood that other communication techniques, either hard-wired or wireless, are also contemplated by the present disclosure as user interface 28. For example, the present disclosure contemplates that user interface 28 may be integrated with a removable storage interface provided by electronic storage 30. In this example, information may be loaded into system 10 from removable storage (e.g., a smart card, a flash drive, a removable disk, etc.) that enables the user(s) to customize the implementation of system 10. Other exemplary input devices and techniques adapted for use with system 10 as user interface 28 comprise, but are not limited to, an RS-232 port, PvF link, an IR link, modem (telephone, cable or other). In short, any technique for communicating information with system 10 is contemplated by the present disclosure as user interface 28.

In some embodiments, electronic storage 30 comprises electronic storage media that electronically stores information. The electronic storage media of electronic storage 30 may comprise one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 30 may comprise one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 30 may store software algorithms, information determined by processor 26, information received via user interface 28, and/or other information that enables system 10 to function properly. Electronic storage 30 may be (in whole or in part) a separate component within system 10, or electronic storage 30 may be provided (in whole or in part) integrally with one or more other components of system 10 (e.g., user interface 28, processor 26, etc.).

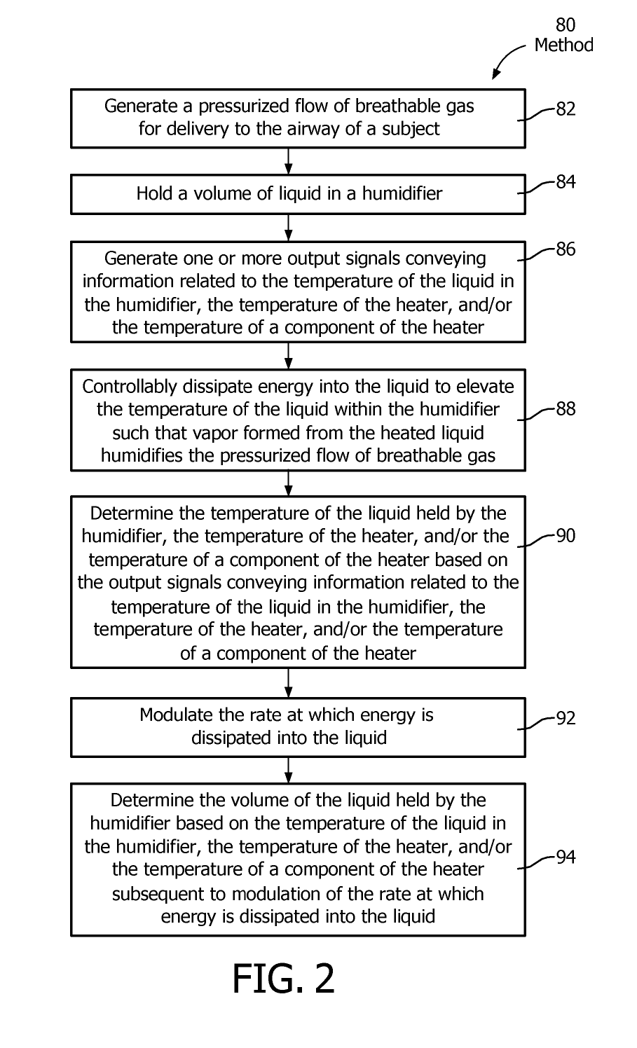

FIG. 2 illustrates a method 80 of generating a pressurized flow of breathable gas for delivery to the airway of a subject. The operations of method 80 presented below are intended to be illustrative. In some embodiments, method 80 may be accomplished with one or more additional operations not described, and/or without one or more of the operations discussed. Additionally, the order in which the operations of method 80 are illustrated in FIG. 2 and described below is not intended to be limiting.

In some embodiments, method 80 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 80 in response to instructions stored electronically on an electronic storage medium. The one or more processing devices may include one or more devices configured through hardware, firmware, and/or software to be specifically designed for execution of one or more of the operations of method 80.

At an operation 82, a pressure generator generates a pressurized flow of breathable gas for delivery to the airway of a subject. In some embodiments, operation 82 is performed by a pressure generator the same as or similar to pressure generator 14 (shown in FIG. 1 and described herein).

At an operation 84, a volume of liquid is held in a humidifier. In some embodiments, operation 84 is performed by a humidifier the same as or similar to humidifier 18 (shown in FIG. 1 and described herein).

At an operation 86, one or more output signals conveying information related to the temperature of the liquid in the humidifier, the temperature of the heater, and/or the temperature of a component of the heater are generated. In some embodiments, operation 86 is performed by sensors the same as humidifier temperature sensors 22 (shown in FIG. 1 and described herein.)

At an operation 88, energy is controllably dissipated into the liquid to elevate the temperature of the liquid within the humidifier such that vapor from the heated liquid humidifies the pressurized flow of breathable gas. In some embodiments, operation 88 is performed by a heater the same as or similar to humidifier heater 20 (shown in FIG. 1 and described herein.)

At an operation 90, the temperature of the liquid held by the humidifier is determined. The liquid temperature determination is based on the output signals conveying information related to the temperature of the liquid in the humidifier, the temperature of the heater, and/or the temperature of a component of the heater. In some embodiments, operation 90 is performed by a processor module the same as temperature module 66 (shown in FIG. 1 and described herein.)

At an operation 92, the rate at which energy is dissipated into the liquid is modulated. In some embodiments, operation 92 is performed by a processor module the same as or similar to humidifier heater control module 68 (shown in FIG. 1 and described herein.)

At an operation 94, the volume of liquid held by the humidifier is determined. The volume determination is based on the temperature of the liquid in the humidifier, the temperature of the heater, and/or the temperature of a component of the heater, subsequent to modulation of the rate at which energy is dissipated into the liquid. In some embodiments, operation 94 is performed by a processor module the same as or similar to liquid volume module 70 (shown in FIG. 1 and described herein.)

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

1. A pressure support system comprising:
a pressure generator configured to generate a pressurized flow of breathable gas for delivery to the airway of a subject;
a humidifier configured to hold a volume of liquid, the humidifier comprising:
one or more temperature sensors configured to generate one or more output signals conveying information related to the temperature of the liquid in the humidifier, the temperature of a heater in the humidifier, and/or the temperature of a component of the heater in the humidifier; and
a heater configured to controllably dissipate energy into the liquid to elevate the temperature of the liquid within the humidifier such that vapor formed from the heated liquid humidifies the pressurized flow of breathable gas; and
one or more processors configured to execute computer program modules, the computer program modules comprising:
a temperature module configured to determine the temperature of the liquid held by the humidifier based on the output signals generated by the one or more temperature sensors;
a heater control module configured to modulate the rate at which energy is dissipated into the liquid by the heater and determine use events during therapy, use events comprising one or more of an expiration of a period of time; an arousal event, or a change in a gas parameter and/or a breathing parameter that indicates wakeful sleep; and
a liquid volume module configured to determine the volume of the liquid held by the humidifier based on the temperature of the liquid in the humidifier, the temperature of the heater, and/or the temperature of a component of the heater, subsequent to modulation of the rate at which energy is dissipated into the liquid by the heater;
wherein after commencement of the therapy, the liquid volume module is configured to determine the volume of the liquid held by the humidifier responsive to a determination of an individual use event by the heater control module.

2. The system of claim 1, wherein the liquid volume module is configured to calculate the slope of the change in liquid temperature, heater temperature, heater component temperature, gas temperature, and/or gas humidity versus time, and to determine the volume of liquid proportional to the slope.

3. The system of claim 1, wherein the heater control module is configured such that modulation of the rate at which energy is dissipated to the liquid comprises an increase in the rate.

4. The system of claim 1, wherein the heater control module and the liquid volume module are configured such that modulation of the rate at which energy is dissipated to the liquid by the heater and the corresponding liquid volume determination are performed at periodic intervals.

5. The system of claim 1, further comprising a user interface module configured to indicate information related to the determined volume of liquid in the humidifier to a user.

6. The system of claim 1, wherein the heater control module is further configured to modulate the rate at which energy is dissipated into the liquid in the humidifier at temperatures lower than the temperature necessary to induce a liquid to gas phase change in the humidifier.

7. A method of generating a humidified, pressurized flow of breathable gas for delivery to an airway of a subject, the method comprising:
generating a pressurized flow of breathable gas for delivery to the airway of the subject;
holding a volume of liquid in a humidifier;
generating one or more output signals conveying information related to a temperature of liquid in the humidifier, a temperature of a heater in the humidifier, and/or a temperature of a component of the heater in the humidifier;
controllably dissipating energy into the liquid to elevate the temperature of the liquid within the humidifier such that vapor formed from the heated liquid humidifies the pressurized flow of breathable gas;
determining the temperature of the liquid held by the humidifier based on the output signals conveying information related to the temperature of the liquid in the humidifier, the temperature of the heater, and/or the temperature of the component of the heater;
modulating the rate at which energy is dissipated into the liquid;
determining the volume of the liquid held by the humidifier based on the temperature of the liquid in the humidifier, the temperature of the heater, and/or the temperature of the component of the heater subsequent to modulation of the rate at which energy is dissipated into the liquid;
determining use events during therapy, use events comprising one or more of an expiration of a period of time, an arousal event, or a change in a gas parameter and/or a breathing parameter that indicates wakeful sleep; and
after commencement of the therapy, determining the volume of the liquid held by the humidifier responsive to a determination of an individual use event.

8. The method of claim 7, further comprising calculating the slope of the change in liquid temperature, heater temperature, and/or heater component temperature versus time, and determining the volume of liquid proportional to the slope.

9. The method of claim 7, wherein modulation of the rate at which energy is dissipated to the liquid comprises an increase in the rate.

10. The method of claim 7, wherein modulation of the rate at which energy is dissipated to the liquid and the corresponding liquid volume determination are performed at periodic intervals.

11. The method of claim 7, further comprising indicating information related to the determined volume of liquid in the humidifier to a user.

12. A pressure support system comprising:
means to generate a pressurized flow of breathable gas for delivery to the airway of a subject;
means to hold a volume of liquid and humidify the pressurized flow of breathable gas, the means to humidify comprising:
means to generate one or more output signals conveying information related to the temperature of the liquid in the means to humidify, the temperature of a means to controllably dissipate energy into the liquid, and/or the temperature of a component of the means to controllably dissipate energy into the liquid; and
means to controllably dissipate energy into the liquid to elevate the temperature of the liquid within the means to humidify such that vapor formed from the heated liquid humidifies the pressurized flow of breathable gas; and
means to execute computer program modules, the computer program modules comprising:
means to determine the temperature of the liquid held by the means to humidify, the means to controllably dissipate energy into the liquid, and/or the component of the means to controllably dissipate energy into the liquid based on the output signals generated by the means to generate output signals conveying information related to the temperature of the liquid in the means to humidify, the temperature of the means to controllably dissipate energy into the liquid, and/or the temperature of the component of the means to controllably dissipate energy into the liquid;
means to modulate the rate at which energy is dissipated into the liquid by the means to controllably dissipate energy into the liquid and determine use events during therapy, use events comprising one or more of an expiration of a period of time, an arousal event, or a change in a gas parameter and/or a breathing parameter that indicates wakeful sleep; and
means to determine the volume of the liquid held by the means to humidify based on the temperature of the liquid in the means to humidify, the temperature of the means to controllably dissipate energy into the liquid, and/or the temperature of the component of the means to controllably dissipate energy into the liquid subsequent to modulation of the rate at which energy is dissipated into the liquid by the means to controllably dissipate energy into the liquid,
wherein, after commencement of the therapy, the means to determine the volume of the liquid is configured to determine the volume of the liquid held by the means to humidify responsive to a determination of au individual use event by the means to modulate and determine use events.

13. The system of claim 12, wherein the means to determine the volume of the liquid held by the means to humidify is configured to calculate the slope of the change in liquid temperature versus time, the change in temperature of the means to controllably dissipate energy into the liquid over time, and/or the change in temperature of the component of the means to controllably dissipate energy into the liquid over time, and to determine the volume of liquid proportional to the slope.

14. The system of claim 12, wherein the means to modulate and determine use events is configured such that modulation of the rate at which energy is dissipated to the liquid comprises an increase in the rate.

15. The system of claim 12, wherein the means to modulate and determine use events and the means to determine the volume of the liquid held by the means to humidify are configured such that modulation of the rate at which energy is dissipated to the liquid and the corresponding liquid volume determination are performed at periodic intervals.

16. The system of claim 12, further comprising means to indicate information related to the determined volume of liquid in the means to humidify to a user.
